(19) United States
(12) Patent Application Publication
(10) Pub. No.: US 2021/0346266 A1
(43) Pub. Date: Nov. 11, 2021

(54) METHOD FOR DYEING A PROTEIN SUBSTRATE

(71) Applicant: Archroma IP GmbH, Reinach (CH)

(72) Inventors: Manuel Jose DOMINGO FARRE, Barcelona (ES); Albert Llort COLELL, Barcelona (ES)

(21) Appl. No.: 17/272,693

(22) PCT Filed: Sep. 3, 2019
(86) PCT No.: PCT/EP2019/073394

§ 371 Date: Mar. 2, 2021

(30) Foreign Application Priority Data
Sep. 4, 2018 (EP) 18192462.2

(51) Int. Cl. A61K 8/46; A61Q 5/10

(52) U.S. Cl. CPC A61K 8/466; A61K 2800/884; A61K 2800/4324; A61Q 5/10

(57) ABSTRACT

The present invention relates to a method for treating at least one protein substrate comprising at least one dyeing step, wherein in the at least one dyeing step at least one dyeing composition comprising at least one sulfur dye is used. The present invention further relates to a hair colorant comprising at least one sulfur dye. It also relates to a protein substrate that has been treated with the method according to the invention or the hair colorant according to the invention. The present invention further relates to the use of a dyeing composition comprising at least one sulfur dye for dyeing at least one protein substrate.

### FIELD OF THE INVENTION

The present invention relates to a method for treating at least one protein substrate comprising at least one dyeing step, wherein in the at least one dyeing step at least one sulfur dye is used. The present invention further relates to a hair colorant comprising at least one sulfur dye. It also relates to a protein substrate that has been treated with the method according to the invention or the hair colorant according to the invention. The present invention further relates to the use of at least one sulfur dye for dyeing at least one protein substrate.

### BACKGROUND OF THE INVENTION

The desire to change the color of protein substrates, in particular of human or animal hair is not a facet of modern times. For a long time human hair has been routinely altered to accommodate the changes of fashion and style. However the attainment of precise initial colors which are retained by the hair for a desirable period has remained a more elusive goal.

Permanent hair colorants should produce end hair colors that are stable for at least 4 to 6 weeks. Additionally, the end hair color should exhibit good wash-fastness, good lightfastness, fastness to rubbing, as well as sufficient resistance with respect to perspiration. Preferably, it should be possible to produce a broad palette of different color shades. Hair colorant formulations also should be stable, for example, in terms of shelf life, ability to retain color benefits after storage and chemical stability both after mixing and during application to hair.

Many compositions for dyeing hair are known. However, most of the human hair colorant compositions are made by employing coloring agents derived from oil derivatives which are considered dangerous for the health and the environment. Most of them are classified as carcinogenic, mutagenic and reprotoxic (CMR) or potential carcinogenic and mutagenic. Examples of these products are p-aminophenol, p-phenylenediamine, resorcinol, m-aminophenol, amino cresols, methyl-, ethyl- or methoxyphenols.

A reaction of these compounds takes place when they are mixed with oxidants such as hydrogen peroxide, persulphates, percarbonates or perborates while they are applied to the hair. Thereby a condensation product is formed that has coloring properties and is responsible for the coloring of the hair. In order to have a good penetration of the hair, these reactions are usually made in ammonia media or using other strong alkaline compounds such as for example ethanolamine.

### OBJECTS OF THE INVENTION

It was therefore an object of the present invention to provide a method for dyeing protein substrates overcoming the drawbacks of the above described dyeing methods, particularly improving the safety and health of human hair dyeing processes. Furthermore it was an object of the present invention to improve environmental compatibility of the dyeing step.

### SUMMARY OF THE INVENTION

The object is solved by a method for treating at least one protein substrate comprising at least one dyeing step, wherein in the at least one dyeing step said protein substrate is applied to an at least one dyeing composition comprising at least one sulfur dye.

In the method according to the invention, the dyeing step can be conducted by applying the at least one sulfur dye to the at least one protein substrate, preferably in form of a hair colorant.

Further, in the method according to the invention, the at least one sulfur dye can be a dye which is obtained by heating organic compounds with sulfur or an alkali polysulfide, wherein the organic compounds are derived from renewable vegetal waste or from petroleum-derived compounds.

Further, in the method according to the invention, the at least one sulfur dye is treated with a reducing agent.

Further, in the method according to the invention, the at least one sulfur dye is used in the thiosulfonic (Bunte salt) form.

Further, in the method according to the invention, the at least one sulfur dye contains less than 5 wt %, preferably less than 1 wt % and more preferably 0 wt % sulfide (Sₓ²⁻, wherein x is at least 1), therein the wt % are based on the weight of the at least one sulfur dye.

Further, in the method according to the invention, the reducing agent is selected from a sugar, hydroxyacetone, sodium thioglycolate, sodium borohydride, thioureadioxide or aldehyde sulfoxilate and combinations thereof.

Further, in the method according to the invention, the dyeing step can last at least 10 seconds, or at least 30 seconds, or at least 1 minute, or at least 5 minutes, or at least 10 minutes, or at least 15 minutes or at least 20 minutes, or at least 25 minutes and at most 40 minutes, or at most 45 minutes, or at most 50 minutes or at most 55 minutes or at most 60 minutes.

Further, in the method according to the invention, the dyeing step can be performed at a temperature of at least 10° C., or at least 15° C., or at least 20° C., or at least 25° C., or at least 30° C., and at most at 35° C., or at most at 40° C.

Further, in the method according to the invention, the method can comprise an oxidation step, which is preferably carried out after the dyeing step.

Further, in the method according to the invention, the oxidation step is accomplished by using hydrogen paroxide or derivatives thereof or air oxidation.

Further, in the method according to the invention the method can comprise a rinsing step with water, and optionally a washing step with a shampoo and/or optionally after-treatment step with a hair-conditioning composition and/or optionally a drying step.

Further, in the method according to the invention the protein substrate can comprise keratin, preferably keratin which is of human origin.

Further, in the method according to the invention, the at least on dyeing composition comprises one or more further dyeing auxiliaries such as solvents, pH-modifying agents, chelants, radical scavengers, conditioning agents, polymer thickener or surfactants.

Another aspect of the invention relates to a dyeing composition, in particular a hair colorant comprising at least one sulfur dye.

Another aspect of the invention relates to protein substrate that has been treated with the method according to the invention or the dyeing composition according to the invention.

Another aspect of the invention relates to the use of the at least one dyeing composition comprising at least one sulfur dye for dyeing at least one protein substrate

### DETAILED DESCRIPTION OF THE INVENTION

The method according to the invention has the advantage of improving the safety and health of protein substrate dyeing processes, particularly the use of sulfur dyes has the advantage that these are non-toxic dyes, i.e. sulfur dyes do not contain metal (ions) or halogens and are considered as polymeric dyes having a high molecular weight and thus as being safe and toxicological harmless.

It is particularly preferred that in the method according to the invention in the at least one dyeing step no dyes other than sulfur dyes are used.

Under the term “protein substrate” within the context of the present application it is to be understood a material to be treated in the at least one dyeing step comprising proteins. Under the term “protein” within the context of the present application biological macromolecules are to be understood, which are formed by peptide bonds of amino acids.

It is particularly preferred that the protein substrate comprises keratin. It is particularly preferred that the protein substrate consists of keratin.

Under the term “keratin” within the context of the present application it is to be understood a collective term for various water-insoluble fiber proteins that are formed by animals or humans, for example hair, nails, horn, hoofs or feathers. “Keratin” within the context of the present application may be regarded as “living,” i.e., on a living body, or as “non-living,” i.e., in a wig, hairpiece, or other aggregation of non-living keratinous fibre. Though mammalian hair is preferred, human hair being a particular example thereof, it will be understood that also wool, fur, and other keratin-containing fibres or mixtures thereof are suitable substrates to be employed in the method according to the invention.

It is particularly preferred that the keratin is of human origin. It is further preferred that the keratin of human origin is human hair.

Under the term “dyeing step” within the context of the present application it is to be understood that the protein substrate is contacted with at least one dyeing composition comprising at least one a dye, in particular a sulfur dye such that the color of the protein substrate after the dyeing step differs from the color of said protein substrate prior to the dyeing step.

It is therefore a preferred aspect of the invention to use at least one dyeing composition comprising at least one sulfur dye for dyeing at least one protein substrate. The advantages of using sulfur dyes are already discussed above and below.

Under the term “dyeing composition”, a composition is to be understood comprising of at least one sulfur dye. The dyeing composition can comprise one or more further dyeing auxiliaries like solvents, pH-modifying agents, chelants, radical scavengers, conditioning agents, polymer thickener or surfactants. In one embodiment, the dyeing composition can consist of at least one sulfur dye. The dyeing composition can be designed as a liquid or as a paste. In any case, the dyeing composition may be applied as such, or may have to be combined with further ingredients like dyeing auxiliaries directly prior to applying the dyeing composition to the protein substrate.

Under the term “applying of the at least one dyeing composition comprising at least one sulfur dye” within the context of the present application any suitable application means or application method is to be understood. For example, the at least one dyeing composition comprising at least one sulfur dye can be sprayed onto the at least one protein substrate or the at least one protein substrate can be contacted with the at least one dyeing composition comprising at least one sulfur dye by dipping the at least one protein substrate into the dyeing composition or the at least one protein substrate can be contacted with the at least one dyeing composition comprising at least one sulfur dye by application of the at least one sulfur dye using a brush, preferably using a tint brush or by application by hands.

It is particularly preferred that the the dyeing step is conducted by applying the at least one dyeing composition comprising at least one sulfur dye to the at least one protein substrate in form of a hair colorant.

In one embodiment, the at least one dyeing composition comprising at least one sulfur dye is applied directly, e.g. applied by using a brush, preferably a tint brush or by hands on the at least one protein substrate to be dyed. The protein substrate can optionally be treated prior to the at least one dyeing step, e.g. mechanically and/or chemically (e.g. to facilitate dye absorption and/or fixation). After the at least one dyeing step, wherein the at least one dyeing composition comprising at least one sulfur dye is applied to the at least one protein substrate, the at least one protein substrate can be further treated, e.g. a rinsing step with water can be accomplished. Optionally a washing step with shampoo and/or optionally a treatment with a conditioning composition and/or a drying step can be accomplished. In one further embodiment the at least one dyeing step can be followed by a further treatment step for enhancing the interaction between the sulfur dye applied in the dyeing step and further treatment compositions like shampoo or conditioning composition. Preferably, the further treatment step is conducted in between the at least one dyeing step and further treatments steps, e.g. rinsing with water, washing with shampoo and/ or optionally treatment with conditioning composition and/ or drying.

Under the term “dyeing solution” within the context of the present application an aqueous solution of the dyeing composition comprising at least one sulfur dye is to be understood. This means that the at least one sulfur dye is at least partly, preferably completely dissolved in a solvent like water and thus, the dyeing solution is formed. In a preferred embodiment, the dyeing solution is designed as a hair colorant.

In a preferred embodiment, the dyeing solution only contains water, at least one dissolved sulfur dye and optionally at least one further agent selected from reducing agents and/or alkaline and/or one or more dyeing auxiliaries as already listed above.

Under the term “sulfur dye” within the context of the present application a dye is to be understood which is obtained by heating organic compounds with sulfur or alkali polysulfide, like for example sodium sulphide (Na₂S) or sodium polysulfide (Na₂Sₓ), wherein the organic raw material compounds can be derived from renewable vegetal waste or from petroleum-derived compounds. The reaction of the organic compound with sulfur or alkali polysulfide is known as sulfurization or thionation. The resulting sulfur dyes cannot be assigned to a specific structural formula. In fact, sulfur dyes can be considered as dyes having a high molecular weight. Under the term “sulfur dye” as used within the context of the present application also a polymeric sulfur dye is to be understood. In the prior art (Colour Index, 3^{rd} edition, Volume 4, published 1971 by the Society of Dyers and Colourists, page 4475 and following which is herewith incorporated by reference) it is described that the dyes obtained by a sulfurization reaction can be controlled by the organic compound selected for sulfurization, conditions of sulfurization and the conditions of the isolation or work-up.

Sulfur dyes derived from petroleum-derived compounds give in particular brown, blue, green, orange, yellow and black colors. Commonly known sulfur dyes derived from petroleum-derived compounds are divided into groups which are determined by the petroleum-derived organic compound predominating in the manufacturing process: 1. Mononuclear benzenoid amine and nitro compounds (Color Index number (in the following also referred to as “CI”) 53005-53135), 2. Binuclear benzenoid amino and nitro compounds (C.I. 53140-53160), 3. Substituted phenols (other than indophenols) (C.I. 53165-53265), 4. Substituted naphthalene (other than indophenols) (C.I. 53270-53300), 5. Polycyclic compounds (C.I. 53320-53335), 6. Indophenols containing (a) two benzene nuclei (C.I. 53400-53442), (b) three benzene nuclei (C.I. 53450-53480), (c) a naphthalene nucleus (C.I. 53520-53590), (d) a carbazole nucleus (C.I. 53630-53640), 7. Acridine, azine, oxazone and thiazone compounds (C.I. 53680-53830). The mentioned sulfur dyes can for example be obtained by sulfurization methods known as sulfur bake, sodium polysulfide bake, sodium polysulfide melt in aqueous and solvent media.

The details of the mentioned manufacturing methods are known to the skilled person. In general, a sulfur bake is the reaction of the raw material (e.g. an amine) is mixed with sulfur or sulfur and sodium sulfide. The mixture is then heated at temperatures of above 200° C. At one point in time the reaction mixture becomes solid. The thus obtained reaction mixture is then dissolved in water and caustic soda and steam boiled for several hours until complete solubilization. The manufacturing method for sulfur dyes in aqueous media is generally conducted as follows: A mixture of the educt, e.g. dinitrophenolate or indophenol is mixed with water and optionally further agents like glycols, glycol-ethers, and/or dispersing agents. The thus obtained mixture is combined with sulfur and sodium sulfide. Thus, sodium polysulfide is obtained which then reacts with the educt. Usually, this reaction is done under reflux at temperatures of between 105 to 160° C. under stirring. Finally, the thus obtained liquid is diluted with more water and, optionally combined with caustic soda and/or reducing agents.

Examples of sulfur dyes originating from petroleum-derived compounds which can be used in the method according to the invention are listed in following table 1.

TABLE 1

| CAS | | |
|---|---|---|
| Registry | | Color |
| Number | Chemical name | shade |
| 66241-11-0 | phenol, 2,4-dinitro-, sulfurized, leuco | Black |
| | derivatives | |
| 70892-34-1 | Formaldehyde, reaction products with | Yellow- |
| | m-phenylenediamine, sodium sulfide | brown |
| | (Na₂S) and sulfur | |
| 85736-99-8 | phenol, 4-(phenylamino)-, sulfurized, | Red-brown |
| | leuco deriv. | |
| 70892-38-5 | Phenol, 4-amino-, reaction products with | Olive- |
| | aniline, 4-methyl-1,3-benzenediamine, | green |
| | p-phenylenediamine, sodium sulfide | |
| | (Na₂S) and sulfur | |
| 90268-98-7 | Carbonic acid disodium salt, reaction | Yellow |
| | products with aniline, 4- | |
| | nitrobenzenamine, p-phenylenediamine, | |
| | sodium sulfide, sulfur and p-toluidine | |
| 100208-66-0 | 1,3-Benzenediamine, 4-methyl-, | Olive- |
| | reaction products with 4- | yellow |
| | nitrobenzenamine, p-phenylenediamine | |
| | and sodium sulfide (Na₂(Sₓ)) | |
| 61902-31-6 | Leuco polysulfided 4-[(2,4- | Dark-blue |
| | dinitrophenyl)amino]phenol | |
| 85940-09-6 | Acetamide, N-(2,4-dinitrophenyl)-, | Olive |
| | reaction products with p- | |
| | phenylenediamine, sodium sulfide | |
| | (Na₂(Sₓ)) and sulfur, leuco deriv. | |
| 12262-25-8 | phenol, 4-[4-(dimethylamino)phenyl] | Blue and |
| | reaction products with sodium sulfide | black |
| | (Na₂Sₓ), leuco derivates | |
| 85940-25-6 | Phenol, 4-(9H-carbazol-3-ylamino)-, | Blue and |
| | reaction products with sodium sulfide | navy |
| | (Na₂(Sₓ)) and sulfur, leuco deriv. | |
| 12262-26-9 | 2,5-Cyclohexadien-1-one, 4-[[4- | Greenish |
| | (phenylamino)phenyl]imino]-, reaction | blue |
| | products with sodium sulfide (Na₂(Sₓ)), | |
| | leuco derivs. | |
| 1040873-93-5 | Phenol, 4-amino-, reaction products with | Reddish blue |
| | 2-ethyl-6-methylbenzenamine, sodium | |
| | hydroxide, sodium sulfide (Na₂S) and | |
| | sulfur | |
| 1326-83-6 | phenol, 2,4-dinitro-, sulfurized, | Black |
| | thiosulfonated | |
| 85940-08-5 | acetamide, N-(2,4-dinitrophenyl)-, | Orange |
| | reaction products with 1-methyl-2,4- | |
| | dinitrobenzene and sodium sulfide | |
| | (Na₂(Sₓ)), leuco derivatives | |
| 71838-68-1 | formamide, N,N′-1,4-phenylenebis-, | Golden |
| | reaction products with 4-methyl-1,3- | yellow |
| | benzenediamine and sulfur, leuco | |
| | derivatives | |
| 68585-53-5 | Thiocyanic acid, (1,3,8,10-tetrahydro- | Red |
| | 1,3,8,10-tetraoxoanthra[2,1,9- | |
| | def:6,5,10-d′e′f′]diisoquinoline-2,9- | |
| | diyl)di-3,1-phenylene ester, reaction | |
| | products with sodium sulfide (Na₂(Sₓ)) | |
| 100208-97-7 | 2-Naphthalenesulfonic acid, 5(or 8)- | Blue |
| | amino-, reaction products with 4- | |
| | aminophenol and sodium sulfide | |
| | (Na₂(Sₓ)) | |
| 12262-32-7 | 1-naphthalenesulfonic acid, 5-[(4- | Green |
| | hydroxyphenyl)amino]-8-(phenylamino)-, | |
| | reaction products with sodium sulfide | |
| | (Na₂(Sₓ)), leuco derivatives | |
| 90480-94-7 | Phenol, 4-amino-, reaction products with | Black |
| | 4-(2-naphthalenylamino)phenol and | |
| | sodium sulfide (Na₂(Sₓ)) | |
| 1040874-53-0 | Disulfurous acid, sodium salt (1:2), | Blue |
| | reaction products with 4-aminophenol, | |
| | 2-ethyl-6-methylbenzenamine, sodium | |
| | sulfide (Na₂S) and sulfur | |

As mentioned before, sulfur dyes can also be obtained by the sulfurization of renewable, vegetal waste. These sulfur dyes are a rather new development. The methods on how to obtain these sulfur dyes are described in WO 2012/163498 and WO 2013/007358.

Renewable, vegetal waste is pretreated under alkaline or acidic conditions and afterwards is treated and reacted with sulfur, sulfides or polysulfide (e.g. S, Na₂S or Na₂Sₓ) at higher temperatures. The thus obtained mixture is then dissolved in aqueous medium and eventually remaining solid particles are removed. In principle, the renewable, agricultural waste can be taken from any kind of plant products, for example from forest or agricultural crop activities or even from marine sources. In particular renewable, vegetal waste suitable to be used as educt for the sulfur dyes can be selected from plant products containing from about 3 to 90% by weight, based on dry mass, of lignin, further components can be fibers or polysaccharides, e.g. cellulose.

Examples of biomass sources obtained from wastes of forestry activities are, e.g. tree barks, wood chips, wood pellets, saw dust, cotton residues, or from agricultural crop activities, such as shells from dry fruits, preferably nutshells, e.g. hazelnut shells, walnut shells, coconut shells, cashew nut shells, pistachio shells, pine fruit shells, and almond shells, further cereals, fruit pits, such as date pits, cherry pits, olive pits. Also plant products, mostly related to fruits, containing phenols and polyphenols in an amount from about 5 to 90% by weight, based on dry mass can be used. Such plant products can further contain components like terpenes, phenolic acids, stilbenes, lignins, flavonoids, tannins, lipids, proteins. Examples are olive pulp, olive orujillo, pips flour, rice residues, grape marc, sun flower pulp, soybean pulp, canola bagasse, herbs ground, corncob and coffee ground. Suitable plant products from marine sources are algae, e.g. brown, red and green algae. Algae usually contain as main components alginic acid, xylans, mannitol, proteins and tannins depending on the algal species.

Examples of sulfur dyes originating from renewable, vegetal waste which can be used in the method according to the invention are further listed in following table 2

TABLE 2

| CAS | |
|---|---|
| Registry Nb. | CA Index name |
| 1456794-36-7 | Oils, rosemary, residues, reaction products with sodium |
| | hydroxide, sodium sulfide (Na₂S) and sulfur |
| 1458581-69-5 | Almond, nutshell, reaction product with Corylus avellana |
| | hazel nutshell, sodium hydroxide, sodium sulfide (NaS₂) |
| | and sulfur |
| 1680199-81-8 | Oils, serenoa repens, residues, reaction products with |
| | sodium hydroxide, sodium sulfide (Na₂S) and |
| | sulfur |
| 1680199-78-3 | Oils, beet, residues, reaction products with sodium |
| | hydroxide, sodium sulfide (Na₂S) and sulfur |
| 1680199-82-9 | Oils, orange, sour, residues, reaction products with |
| | sodium hydroxide, sodium sulfide (Na₂S) and |
| | sulfur |

The sulfur dyes can be used in the thiosulfonic (Bunte salt) form.

In a preferred embodiment the at least one sulfur dye has been treated with a reducing agent in order to obtain the water soluble leuco form of the sulfur dye which is then contacted with the at least one protein substrate. Consequently, the term “sulfur dye” as used within the context of the present application also encompasses the water soluble form thereof, i.e. the leuco sulfur dye. The treatment of the sulfur dye with the reducing agent can be done prior to the dyeing step. For example, there are pre-reduced sulfur dyes available wherein the sulfur dyes are sold in their respective leuco form and can be readily applied in the method according to the invention. Such pre-reduced sulfur dyes are for example sold by Archroma under the trade name Diresul. The sulfide (Sₓ²⁻, wherein x is at least 1) content of the pre-reduced sulfur dyes should be less than 5 wt %, or less than 3 wt %, or less than 1 wt %, preferably 0 wt %, wherein the wt % are based on the weight of the pre-reduced sulfur dye. The treatment of the sulfur dye can also be done during the dyeing step, i.e. the water-insoluble sulfur dye, e.g. used in form of a powder or liquid wherein the sulfur dye is present in its oxidized form is combined with water and the reducing agent. Thus, the water-insoluble sulfur dye is reduced into its water-soluble leuco form due to the reaction with the reducing agent present in the water. Another possibility is to use solubilized sulfur dyes. Solubilized sulfur dyes are thiosulfonic acid derivatives of sulfur dyes. Solubilized sulfur dyes are water soluble and can be obtained by treating the water-insoluble sulfur dye with sulfite or bisulfite agents.

It is possible to use as reducing agents sugars, like dextrose, glucose, fructose, hydroxyacetone, sodium thioglycolate, sodium borohydride, thiourea dioxide or aldehyde sulfoxylate, sodium hydrosulfite, or combinations thereof can be used, because these types of reducing agents are safe and environmental friendly and the dyeing solution can be readily disposed and do not need any special treatment in order to remove the reducing agent. Particularly preferred reducing agents are sugars, like dextrose, glucose, fructose and sodium thioglycolate.

The amount of reducing agent used, respectively if it is necessary at all to add at least one reducing agent to the dyeing composition depends on several factors like duration of the dyeing step, equipment used, or dye concentration in the dyeing composition. Conventionally used amounts of the at least one reducing agent used in a dyeing composition are 0 g/I up to at most 250 g/I.

The advantasddddddge of using sulfur dyes instead of other dyes like basic or metallized dyes is that sulfur dyes do not contain metal (ions) or halogens, which are often regarded as harmful compounds. Further, the sulfur dyes employed are present in the form of a dye and there is no need to perform the coupling reaction during application of the components to the at least protein substrate. The sulfur dyes can be regarded as non-toxic dyes. Further, since sulfur dyes can be considered as polymeric dyes having a high molecular weight, and are therefore regarded as safe and toxicological harmless. Using sulfur dyes has the further advantage that the dyeing composition comprising at least one sulfur dye can be stored for further use for other dyeing steps, due to the high stability of the sulfur dyes in their leuco form present in the dyeing composition. This is particularly advantageous if for example wigs made of human hair are dyed.

Sulfur dyes are commonly employed in dyeing cellulosic textiles. However, sulfur dyes have yet not been used in dyeing materials other than cellulosic textiles. With the present application it was successfully achieved to dye protein substrates, e.g. wool or human hair using a dyeing composition comprising at least one sulfur dyes.

In one embodiment the method according to the invention further comprises the use of an alkaline compound in the at least one dyeing step. Alkaline compounds are used in order to adjust the pH of the composition containing the at least one sulfur dye. By using alkaline compounds it can be ensured that the pH of the dyeing composition is in such pH range that the at least one dye is able to penetrate the at least one protein substrate and to be fixed on the at least one protein substrate. Examples for alkaline compounds which can be used are potassium carbonate, ammonia or organic alkaline compounds like ethanolamine, diethanolamine, triethanolamin, triethylenetetramine.

The dyeing step is performed at a temperature of at least 10° C., or at least 15° C., or at least 20° C., or at least 25° C., or at least 30° C., and at most at 35° C., or at most at 40° C. Preferably, the dyeing step is performed at a temperature of at least 15° C. and at most 30° C., or at least 20° C. and at most 25° C. Preferably the dyeing step is performed at ambient temperature, which is within the context of the present application to be understood a temperature of at least 20° C. and at most 25° C. It is preferred that the dyeing is performed without the supply of external heat.

In one embodiment the dyeing step lasts at least 10 seconds, or at least 30 seconds, or at least 1 minute, or at least 5 minutes, or at least 10 minutes, or at least 15 minutes, or at least 20 minutes, or at least 25 minutes, and at most 40 minutes, or at most 45 minutes, or at most 50 minutes, or at most 55 minutes, or at most 60 minutes. The duration of the dyeing step is this time range during which the at least one protein substrate is contacted with the at least one dyeing composition comprising at least one sulfur dye. If for example a pale color tone of the at least one protein substrate should be achieved, it is enough to contact said protein substrate with the at least one dyeing composition comprising at least one sulfur dye for only some minutes. If, however, a more intense color tone of the at least one protein substrate should be achieved the dyeing step can last up to one hour.

In one embodiment the method according to the invention comprises a rinsing step with water. Optionally a washing step with shampoo and/or optionally an after-treatment step with a conditioning composition and/or a drying step can be accomplished.

It is also possible to employ an oxidizing step into the method according to the invention. In a preferred embodiment, the oxidizing step is conducted after the dyeing step and is particularly employed to the at least one protein substrate which is dyed in the dyeing step. This has the advantage that the applied sulfur dye, which is present in its leuco form after the dyeing step is oxidized and thus turned into the water-insoluble sulfur dye form having the finally desired and stable coloration. For example if a composition comprising a sulfur dye with the chemical name “phenol, 4-[4-(dimethylamino)phenyl] reaction products with sodium sulfide (Na₂Sₓ), leuco derivates” registered under the CAS registry number 12262-25-8 (see also table 1) is used, the dyed surface of the hair has a greenish appearance after the dyeing step due to the presence of the leuco form of said sulfur dye. After the oxidizing step, the color changes to a deep bluish-black, since the leuco form is oxidized and thus, the water-insoluble sulfur dye having the deep bluish-black color is obtained. The oxidization step can be conducted by any suitable means, e.g. by a washing step with the presence of small quantities of oxidant or by air oxidation in a drying step. Suitable oxidizing agents are for example hydrogen peroxide or derivatives thereof or air. Oxidizing agents are used in an amount of at least 0.1 g/I, or at least 1g/I, or at least 5 g/I, or at least 10 gr/l, or at least 20 g/I, or at least 30 g/I, or at least 40 g/I, or at least 50 g/I, or at most 60 g/I, or at most 70 g/I, or at most 80 g/I, or at most 90 g/I, or at most 100 g/I. Preferably, oxidizing agents are used in an amount of at least 0.1 g/I to at most 100 g/I, or at least 5 g/I to at most 50 g/I, or at least 10 g/I to at most 30 g/I. Oxidizing agents can be combined with oxidizing catalysts to increase the speed of the oxidizing step. Suitable oxidizing catalysts are e.g. acidic organic metal complexes. Suitable oxidizing agents are for example peroxides, perborates, persulfates, percarbonates.

In a preferred embodiment of the method according to the invention, the at least one dyeing step is followed by at least one oxidizing step. In one further embodiment of the method according to the invention, a further treatment step for enhancing the interaction between the sulfur dye and further treatment compositions, i.e. shampoo, conditioner compositions, oxidizing agents, is conducted in between the at least one dyeing step and the at least one oxidizing step.

Optionally, the at least one dyeing step is followed by at least one washing step and by at least one oxidizing step. In one further embodiment of the method according to the invention, a further treatment step for enhancing the interaction between the sulfur dye and further treatment compositions, i.e. shampoo, conditioner compositions, oxidizing agents, is conducted in between the at least one dyeing step and the at least one washing step and the at least one oxidizing step.

In another preferred embodiment of the method according to the invention, the at least one dyeing step is followed by at least one oxidizing step and at least one washing step. In one further embodiment of the method according to the invention, a further treatment step for enhancing the interaction between the sulfur dye and further treatment compositions, i.e. shampoo, conditioner compositions, oxidizing agents, is conducted in between the at least one dyeing step and the at least one oxidizing step and the at least one washing step.

In another preferred embodiment of the method according to the invention, the following steps are carried out, preferably in the given order: a) washing the protein substrate and optionally thereby preparing same for dyeing, b) dyeing, c) oxidizing, d) washing, e) drying the at least one protein substrate. The washing step d) can encompass at least one rinsing step using water and/or shampoo and/or a conditioning step. In one further embodiment of the method according to the invention the further treatment step for enhancing the interaction between the sulfur dye and further treatment compositions, i.e. shampoo, conditioner compositions, oxidizing agents, is conducted between step b) and c).

Under the term “shampoo” within the context of the present application a composition is to be understood which is used for cleaning the protein substrate, in particular hair. Usually, shampoo is applied to the protein substrate, in particular to the wet protein substrate, which is preferably wet hair, massaging the product into the same and then rinsing it off by using water.

In a second aspect the present invention further relates to a dyeing composition, in particular a hair colorant comprising at least one sulfur dye. Thus, according to this aspect, the dyeing composition is designed as hair colorant.

The term “hair colorant” or “hair colorant composition” within the context of the present application denotes a dyeing composition that is suitable to be applied to a protein substrate as defined above, in particular to human hair and is able to change the color of the substrate. The hair colorant composition can be designed as liquid or as paste. In any case, the hair colorant composition according to the invention may be applied as such, or may have to be combined with further ingredients like dyeing auxiliaries directly prior to applying the hair colorant to the protein substrate.

The at least one sulfur dye of the hair colorant according to the invention is a dye which is obtained by heating organic compounds with sulfur or alkali polysulfide, wherein the organic compounds can be derived from renewable vegetal waste or from petroleum-derived compounds. All previous specifications of the at least one sulfur dye employed in the method according to the invention apply accordingly to the at least one sulfur dye which is present in the hair colorant according to the invention.

Preferably, the at least one sulfur dye of the hair colorant according to the invention has been treated with a reducing agent. All previous specifications of the reducing agent for treatment of the at least one sulfur dye employed in the method according to the invention apply accordingly to the reducing agent for treatment of the at least one sulfur dye which is present in the hair colorant according to the invention.

The hair colorant composition may contain additional components, in particular dyeing auxiliaries as already listed with reference to the dyeing composition that may be selected from those known in the art.

The hair colorant composition may comprise solvents such as water, lower aliphatic alcohols, for example aliphatic alcohols with from 1 to 4 carbon atoms such as ethanol, propanol and isopropanol, aromatic alcohols such as benzyl alcohol and phenoxyethanol or polyols or poly ethers such as glycerin and 1,2- and 1,3-propyleneglycol, carbitol, 2-butoxyethanol, diethylene glycol, monoethyl ether, monomethyl ether, hexylene glycol, ethylene glycol, ethoxy glycol, butoxydiglycol, ethoxydiglycerol, dipropyleneglycol and polyglycerol.

The hair colorant composition may comprise a pH-modifying agent such as an alkalizing agent.

Any alkalizing agent known in the art may be used such as: alkanolamines, for example monoethanolamine, diethanolamine, triethanolamine, monopropanolamine, dipropanolamine, tripropanolamine, 2-amino-2-methyl-1,3-propanediol, 2-amino-2-methyl-1-propanol, and 2-amino-2-hydroxymethyl-1,3-propanediol; guanidium salts; and alkali-metal and ammonium hydroxides and carbonates, such as sodium hydroxide and ammonium carbonate. Particularly, preferred alkalizing agents are those which provide a source of ammonium ions. Any source of ammonium ions is suitable for use herein. Preferred sources of ammonium ions include ammonium chloride, ammonium sulfate, ammonium nitrate, ammonium phosphate, ammonium acetate, ammonium carbonate, ammonium hydrogen carbonate, ammonium carbamate, ammonium hydroxide, percarbonate salts, ammonia and mixtures thereof. Particularly preferred are ammonium carbonate, ammonium carbamate, ammonia, and mixtures thereof. The hair colorant composition may comprise from 0.1% to 10% by weight, such as from 0.5% to 5%, such as from 1% to 3% of an alkalizing agent.

The hair colorant compositions may have a pH of from 7 to 12, preferably from 8 to 11, preferably from 7.5 to 9.5, even more preferably from 8.4 to 9.5, most preferably from 8.5 to 9.4, for example, 9.0 or 9.3.

The hair colorant composition may optionally contain chelants. Suitable optional chelants for use in hair colorant compositions are carboxylic acids (in particular aminocarboxylic acids) and phosphonic acids (in particular aminophosphonic acids) and polyphosphoric acids (e.g., linear polyphosphoric acids), including the salts and derivatives of these chelants. Generally, the chelants do not penetrate the hair to any significant extent under typical oxidative hair coloring conditions. As such, the chelants generally do not affect color formation inside the hair.

The hair colorant compositions may comprise from 0.01% to 5%, preferably from 0.25% to 3%, preferably from 0.5% to 1% of chelant, salts thereof, derivatives thereof, or mixtures thereof.

The hair colorant compositions may comprise an aminocarboxylic acid chelant. Aminocarboxylic acid chelants as defined herein are chelants having at least one carboxylic acid moiety (—COOH) and at least one nitrogen atom. Examples of aminocarboxylic acid chelants suitable for use herein include diethylenetriamine pentaacetic acid (DTPA), ethylenediamine disuccinic acid (EDDS), ethylenediamine diglutaric acid (EDGA), 2-hydroxypropylenediamine disuccinic acid (HPDS), glycinamide-N,N′-disuccinic acid (GADS), ethylenediamine-N, N′-diglutaric acid (EDDG), 2-hydroxypropylenediamine-N, N′-disuccinic acid (HPDDS), ethylenediaminetetraacetic acid (EDTA), ethylenedicysteic acid (EDC), ethylenediamine-N, N′-bis(o-hydroxyphenyl acetic acid) (EDDHA), diem inoalkyldi(sulfosuccinic acids) (DDS), N,N′-bis(2-hydroxybenzyl)ethylenediamine-N,N′-diacetic acid (HBED), salts thereof and derivatives thereof.

Other suitable aminocarboxylic type chelants for use herein are iminodiacetic acid derivatives such as N-2-hydroxyethyl-N,N diacetic acid or glyceryl imino diacetic acid, iminodiacetic acid-N-2-hydroxypropyl sulfonic acid and aspartic acid N-carboxymethyl N-2-hydroxypropyl-3-sulfonic acid, 13-alanine-N,N′-diacetic acid, aspartic acid-N,N′-diacetic acid, aspartic acid-N-monoacetic acid and iminodisuccinic acid chelants, ethanoldiglycine acid, salts thereof and derivatives thereof. Dipicolinic acid and 2-phosphonobutane-1,2,4-tricarboxylic acid are also suitable.

The hair colorant compositions may comprise from 0.1% to 5% of diethylenetriaminepenta(methylenephosphonic acid) and from 0.1% to 5% of ethylenediamine-N,N′-disuccinic acid, and from 0.1% to 5% of diethylenetriaminepentaacetic acid. Preferred for use herein is ethylenediamine-N,N′-disuccinic acid (EDDS), derivatives and salts thereof.

The hair colorant compositions may comprise a chelant selected from aminophosphonic acid type chelants, salts thereof, derivatives thereof and mixtures thereof. Amino-phosphonic acid type chelants are defined as chelants comprising an aminophosphonic acid moiety (—PO₃H₂) or its derivative —PO₃R₂ wherein R₂ is a C₁ to C₆ alkyl or aryl radical.

Suitable aminophosphonic acid type chelants for use herein are aminotri-(1-ethylphosphonic acid), ethylenediaminetetra-(1-ethylphosphonic acid), aminotri-(1-propylphosphonic acid), and aminotri-(isopropylphosphonic acid). Preferred chelants for use herein are aminotri-(methylenephosphonic acid), ethylenediaminetetra(methylenephosphonic acid) (EDTMP) and diethylene-triamine-penta-(methylenephosphonic acid) (DTPMP) and mixtures thereof.

The hair colorant compositions may further comprise a source of radical scavenger. As used herein, the term “radical scavenge”refers to a species that can react with a radical, e.g. a carbonate radical, to convert the radical by a series of fast reactions to a less reactive, or unreactive, species.

Suitable radical scavengers for use herein may be selected from the classes of alkanolamines, amino sugars, amino acids, esters of amino acids, pyrazolones, such as those discussed in US 2011/0035885A1 and US 2011/0035886A1, and mixtures thereof. Particularly preferred compounds are: monoethanolamine, 3-amino-1-propanol, 4-amino-1-butanol, 5-amino-1-pentanol, 1-amino-2-propanol, 1-amino-2-butanol, 1-amino-2-pentanol, 1-amino-3-pentanol, 1-amino-4-pentanol, 3-amino-2-methylpropan-1-ol, 1-amino-2-methylpropan-2-ol, 3-aminopropane-1,2-diol, glucosamine, N-acetylglucosamine, glycine, arginine, lysine, proline, glutamine, histidine, sarcosine, serine, glutamic acid, tryptophan, and mixtures thereof, and the salts such as the potassium, sodium and ammonium salts thereof, 3-carboxy-1H-pyrazol-5-one, 3-carboxy-1-phenyl-pyrazol-5-one, 3-carboxy-1-(4-sulfophenyI)-pyrazol-5-one, 3-carboxy-1-(4-carboxyphenyl)-pyrazol-5-one, and mixtures thereof. Preferred compounds are glycine, sarcosine, lysine, serine, 2-methoxyethylamine, glucosamine, glutamic acid, morpholine, piperidine, ethylamine, 3-amino-1-propanol, and mixtures thereof. The hair colorant compositions may comprise from 0.1% to 10% by weight, preferably from 1% to 7% by weight of the hair colorant composition of a radical scavenger.

The hair colorant composition may comprise a conditioning agent. Optionally, a separate conditioning composition comprising a conditioning agent may be used after the hair colorant composition. Conditioning agents suitable are selected from silicone materials, aminosilicones, fatty alcohols, polymeric resins, polyol carboxylic acid esters, cationic polymers, insoluble oils and oil derived materials and mixtures thereof. Additional materials include mineral oils and other oils such as glycerin and sorbitol. Particularly useful conditioning materials are cationic polymers. Conditioners of cationic polymer type can be chosen from those comprising units of at least one amine group chosen from primary, secondary, tertiary and quaternary amine groups that may either form part of the main polymer chain, or be borne by a side substituent that is directly attached to the main polymer chain. Silicones can be selected from polyalkylsiloxane oils, linear polydimethylsiloxane oils containing trimethylsilyl or hydroxydimethylsiloxane endgroups, polymethylphenylsiloxane, polydimethylphenylsiloxane or polydimethyldiphenylsiloxane oils, silicone resins, organofunctional siloxanes having in their general structure one or a number of organofunctional group(s), the same or different, attached directly to the siloxane chain or mixtures thereof. Said organofunctional group(s) are selected from: polyethyleneoxy and/or polypropyleneoxy groups, (per)fluorinated groups, thiol groups, substituted or unsubstituted amino groups, carboxylate groups, hydroxylated groups, alkoxylated groups, quaternium ammonium groups, amphoteric and betaine groups. The silicone can either be used as a neat fluid or in the form of a pre-formed emulsion. In one further embodiment of the method according to the invention, the separate conditioning composition comprising above described conditioning agents may be applied in a treatment step for enhancing the interaction between the sulfur dye and further treatment compositions, i.e. shampoo, conditioner compositions, oxidizing agents. Preferable the treatment step is conducted in between the at least one dyeing step and the at least one or more further steps such as a washing step and the at least one oxidizing step.

The conditioning agent generally will be used at levels of from 0.05% to 20% by weight of the hair colorant composition or the conditioning composition, such as from 0.1% to 15%, such as from 0.2% to 10%, such as from 0.2% to 2% by weight of the hair coloring composition or the conditioning composition.

The hair colorant composition may comprise a polymer thickener, comprising at least one polymer selected from associative polymers, polysaccharides, non-associative polycarboxylic polymers, and mixtures thereof.

The hair colorant may comprise one or more surfactants. Surfactants suitable for use herein generally have a lipophilic chain length of from 8 to 30 carbon atoms and can be selected from anionic, nonionic, amphoteric and cationic surfactants and mixtures thereof. In one embodiment, the total level of surfactant is from 1% to 60%, or from 2% to 30%, or from 8% to 25%, or from 10% to 20% by weight.

Application of the hair colorant according to the invention to the protein substrate may be undertaken in several ways known in the art. In case the protein substrate is hair, the application of the hair colorant composition may take place on the whole head of hair of an end user. By “whole head of hair” it is meant that the hair all over the head from the root of the hair to the tip of the hair is included in the application process. By contrast, the application of the hair colorant composition may take place on the root portion of the hair. The application to the root portion of the hair may still be over the entire head of the end user, but application of the hair colorant composition is applied only to the section of hair closest to the head (root portion), which is between about 0.01 mm to about 4 mm from the scalp of the head. Also, application may take place on a portion of hair. Application to a portion of hair is commonly referred to as highlighting or lowlighting. The portion of hair may be physically separated from the whole head of hair in a hair bundle or may be a smaller portion of hair than the whole head of hair. A hair bundle may be physically separated from a whole head of hair by a device including a plastic cap through which hair bundles are formed when hair is pulled through orifices in the plastic cap, metal foils encompassing a hair bundle, strand separators applied to hair at the root portion, or similar devices. The described application varieties with reference to hair of an end user, of course also applies to all other herein defined protein substrates, like for example artificial hair for wigs.

The hair colorant according to the invention might be worked into the protein substrate by hand or by a tool for a few minutes to ensure uniform. The hair colorant composition remains on the protein substrates while the end color develops for a time period of 1 to 60 minutes. The hair colorant is then rinsed off thoroughly with tap water and the protein substrate is allowed to dry and/or is styled.

In a third aspect the present invention relates to a protein substrate that has been treated with the method according to the invention or the dyeing composition, in particular the hair colorant according to the invention.

In one embodiment the protein substrate comprises keratin. It is particularly preferred that the protein substrate consists of keratin. Keratin was already defined above with reference to the method according to the invention.

It is particularly preferred that the keratin is of human origin. It is further preferred that the keratin of human origin is human hair.

In a fourth aspect the present invention relates the use of at least one dyeing composition comprising at least one sulfur dye for dyeing at least one protein substrate. All specification made herein for the method according to the invention, the hair colorant according to the invention and the protein substrate according to the invention accordingly apply the use of at least one dyeing composition comprising at least one sulfur dye for dyeing at least one protein substrate.

### EXAMPLES

The method according to the invention is in the following described in more detail. However, the method according to the invention should not be limited to the following examples.

### Example 1

An aqueous solution is prepared containing 20 wt % of a sulfur dye registered under the CAS registry number 12262-25-8 (see Table 1), 10 wt % of an 25% aqueous sodium thioglycolate solution and 3 wt % of ammonia (wt % are based on the weight of the aqueous solution).

A piece of hair (Yak hair) is dipped into this solution for 45 minutes. Afterwards the piece of hair is washed with warm water eliminating the non-fixed dye, and finally is dipped in another solution containing 5wt %of a solution of hydrogen peroxide which concentration is 30%for 10 minutes.

A deep and brilliant colored black hair is obtained, which is fast to wash and light.

### Example 2

An aqueous solution is prepared containing 10 wt % of a sulfur dye registered under CAS registry number 1456794-36-7 (see table 2) 10 wt % of an 25% aqueous sodium thioglycolate solution and 3 wt % of ammonia (wt % are based on the weight of the aqueous solution).

A piece of hair (Yak hair) is dipped into this solution for 45 minutes. Afterwards the piece of hair is washed with warm water eliminating the non-fixed dye, and finally is dried with hot air for 5 minutes

A medium and brilliant coloured brown hair is obtained, which is fast to wash and light.

### Example 3

A hair colorant containing 15 wt % of a sulfur dye registered under CAS registry number 1456794-36-7 (see table 2)10 wt % of an 25% aqueous sodium thioglycolate solution and 3 wt % of Ammonia is prepared mixing the previous aqueous solution with a composition comprising 35 wt % laureth-2, 20 wt % propylene glycol, 15% Lauryl Sulphate, 8 wt % monoethanolamine, 7 wt % stearic acid, 5 wt % sodium heptagluconate and 10 wt % water (wt % are based on the weight of the aqueous solution or composition).

A piece of hair (Yak hair) is dipped into the hair colorant for 45 minutes. Afterwards the piece of hair is washed with warm water eliminating the non-fixed dye, and finally is dried with hot air for 5 minutes

A medium and brilliant coloured brown hair is obtained, which is fast to wash and light.

### Example 4

An aqueous solution is prepared containing 20 wt % of a sulfur dye registered under CAS registry number 1456794-36-7 (see table 2), 10 wt % of an 25% aqueous sodium thioglycolate solution and 3 wt % of ammonia (wt % are based on the weight of the aqueous solution).

A piece of hair (Yak hair) is dipped into this solution for 45 minutes. Afterwards the piece of hair is washed with warm water eliminating the non-fixed dye, and finally is dipped into a solution containing 5 wt % of hydrogen peroxide which concentration is 30% for 10 minutes.

A deep and brilliant coloured brown hair is obtained which is fast to wash and light.

### Example 5

An aqueous solution is prepared containing 20 wt % of a sulfur dye registered under CAS registry number 1456794-36-7 (see table 2), 10 wt % of an 25% aqueous sodium thioglycolate solution, and 5 wt % of potassium carbonate (wt % are based on the weight of the aqueous solution).

A piece of hair (Yak hair) is dipped into this solution for 45 minutes. Afterwards the piece of hair is washed with warm water eliminating the non-fixed dye, and finally is dipped into a solution containing 5 wt % of hydrogen peroxide which concentration is 30% for 10 minutes.

A medium-high and brilliant colored brown hair is obtained (showing less intensity than in example 3), which is fast to wash and light.

### Example 6

An aqueous solution is prepared containing 20 wt % of sulfur dye registered under CAS registry number 12262-32-7 (see table 2), 10 wt % of an 25% aqueous sodium thioglycolate solution and 3 wt % of ammonia (wt % are based on the weight of the aqueous solution).

A piece of hair (Yak hair) is dipped into this solution for 45 minutes. Afterwards the piece of hair is washed with warm water eliminating the non-fixed dye, and finally is dipped into a solution containing 5 wt % of hydrogen peroxide which concentration is 30% for 10 minutes.

A deep dark greenish black colored hair is obtained, which is fast to wash and light.

### Example 7

A hair colorant containing 20 wt % of a sulfur dye registered under CAS registry number 12262-32-7 (see table 1) 10 wt % of an 25% aqueous sodium thioglycolate solution, and 3 wt % of ammonia. is prepared mixing the previous aqueous solution with a composition comprising 30 wt % laureth-2, 15 wt % propylene glycol, 5 wt % cetearylalcohol, 15 wt % lauryl sulphate, 10 wt % diethanolamine, 7 wt % citric acid, 5 wt % sodium heptagluconate, 3 wt % silicone derivative and 10 wt % water (wt % are based on the weight of the aqueous solution or composition).

A piece of hair (Yak hair) then is impregnated with this mixture and maintained during 45 minutes. Afterwards the piece of hair is washed with warm water eliminating the non-fixed dye, and finally is immersed in a solution containing 5% of hydrogen peroxide which concentration is 30% during 10 minutes.

A greenish grayish colored hair is obtained, which is fast to wash and light.

1. A method for treating at least one protein substrate comprising at least one dyeing, wherein in the at least one dyeing, said at least one protein substrate is applied to an at least one dyeing composition comprising at least one sulfur dye.

2. The method according to claim 1, wherein the at least one sulfur dye is a dye which is obtained by heating one or more organic compounds with sulfur or an alkali polysulfide, wherein the one or more organic compounds are derived from renewable vegetal waste or from one or more petroleum-derived compounds.

3. The method according to claim 1 wherein the at least one sulfur dye is used in a thiosulfonic (Bunte salt) form.

4. The method according to claim 1 wherein the at least one sulfur dye comprises less than 5 wt % optionally less than 1 wt % and optionally 0 wt %, sulfide (Sₓ²⁻ wherein x is at least 1) wherein the wt % are based on the weight of the at least one sulfur dye.

5. The method according to claim 1, wherein the at least one sulfur dye has been treated with a reducing agent, wherein the reducing agent is optionally selected from a sugar, hydroxyacetone, sodium thioglycolate, sodium borohydride, thiourea dioxide or aldehyde sulfoxylate, and combinations thereof.

6. The method according to claim 1, wherein the dyeing lasts at least 10 seconds, or at least 30 seconds, or at least 1 minute, or at least 5 minutes, or at least 10 minutes, or at least 15 minutes or at least 20 minutes, or at least 25 minutes and at most 40 minutes, or at most 45 minutes, or at most 50 minutes or at most 55 minutes or at most 60 minutes.

7. The method according to claim 1, wherein the dyeing [[step]] is performed at a temperature of at least 10 ° C., or at least 15 ° C., or at least 20 ° C., or at least 25 ° C., or at least 30 ° C., and at most at 35 ° C., or at most at 40 ° C.

8. The method according to claim 1, wherein the method comprises an oxidation, which is optionally carried out after the dyeing, or wherein at least one treatment is conducted between the at least one dyeing and the at least one oxidizing.

9. The method according to claim 8, wherein the oxidation is accomplished by using hydrogen peroxide or a derivative thereof or air oxidation.

10. The method according to claim 1, wherein the method comprises rinsing with water, and optionally washing with a shampoo and/or optionally an after-treatmeat with a conditioning composition and/or optionally drying.

11. The method according to claim 1, wherein the protein substrate comprises keratin, optionally keratin which is of human origin.

12. The method according to claim 1, wherein the dyeing composition comprises one or more further dyeing auxiliaries, optionally solvents, pH-modifying agents, chelants, radical scavengers, conditioning agents, polymer thickener or surfactants

13. A dyeing composition, optionally hair colorant, comprising at least one sulfur dye.

14. A protein substrate that has been treated with the method according to claim 1 or a dyeing composition comprising at least one sulfur dye.

15. A product comprising at least one dyeing composition comprising at least one sulfur dye for dyeing at least one protein substrate.
